(19) United States
(12) Patent Application Publication
(10) Pub. No.: US 2008/0051782 A1
(43) Pub. Date: Feb. 28, 2008

(54) Auxiliary apparatus for a vertebra surgical operation

(76) Inventor: Shing-Sheng Wu, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/497,312

(22) Filed: Aug. 2, 2006

(51) Int. Cl. A61F 2/30

(52) U.S. Cl. 606/61

(57) ABSTRACT

An auxiliary apparatus for a vertebra surgical operation is applied to a small incision surgical operation. The auxiliary apparatus includes an auxiliary part having a regulating opening, a guiding block attached on the rear side of the auxiliary part, a plurality of sleeves regulated by the regulating opening, and a puncturing part passing through the guiding block and the lower side of the sleeves. The sleeves are placed in expanding parts and the expanding parts are placed into the muscle. There are through holes located at the jointing locations of the bone screws and the sleeves. One end of the puncturing part is connected with a linking rod. The puncturing part passes through the guiding block. One end of the linking rod punctures into muscle and passes through the expanding parts and the through holes to receive the linking rod in the plow grooves of the bone screws.

### BACKGROUND OF THE INVENTION

1. Field of the Invention

The present invention relates to an auxiliary apparatus for a vertebra surgical operation. In particular, this invention relates to an auxiliary apparatus for a small incision vertebra surgical operation.

2. Description of the Related Art

In traditional vertebra surgical operations, the surgeon needs to open a large incision on the patient's back to view the patient's vertebra. In order to open a large incision, a large area of the back muscle has to be separated from the vertebra. Therefore, the patient requires a long time to recover to his or her normal condition. Meanwhile, the pain produced by separating the muscle from the vertebra in the surgical operation has to be lowered by the use of an anodyne. The patient runs the risk of side effects caused by the anodyne, and requires a long time to convalesce. Because the muscle attached on the vertebra is separated from its original location, there are scars after the muscle has healed. A scar produced between two muscle layers often causes each of the muscle layers to lose their independent function. Furthermore, separating the muscle sometimes causes innervation and muscular atrophy. It leads to a weakening of the dorsum muscles from which few patients ever fully recover.

Other problems also occur, for example, the dorsum muscles become tired and ache, causing the patient physical discomfort, and the patient's physical exertion and movement is limited.

A vertebra surgical operation that involves a smaller incision overcomes many of the problems described above. A smaller incision vertebra surgical operation has many positive characteristics, such as reducing the complexity of the surgical procedure, reducing blood lose during the operation, reducing the need for extended use of anodyne, shortening the required period for convalescence, and a decreasing of the negative affects upon the patient's physical strength so that they may return to their daily activities as quickly as possible. Therefore, appliances and auxiliary apparatuses that enable a smaller incision surgical operation are developing to make progress in the surgical operation.

In order to own the knowledge and ability of the surgical operation using a smaller incision, a traditional surgical operation and clinical experience are required. In the surgical operation, a proper auxiliary apparatus is necessary.

### SUMMARY OF THE INVENTION

One particular aspect of the present invention is to provide an auxiliary apparatus for a vertebra surgical operation. This invention especially relates to an auxiliary apparatus for a small incision vertebra surgical operation. It is convenient for a surgeon to execute such a vertebra surgical operation and the goals of the small incision surgical operation are achieved, including making a small incision that will heal quickly.

The auxiliary apparatus for a vertebra surgical operation of the present invention is applied to a small incision vertebra surgical operation. The auxiliary apparatus for a vertebra surgical operation includes an auxiliary part having a regulating opening, a guiding block attached on the rear side of the auxiliary part, a plurality of sleeves regulated by the regulating opening, and a puncturing part passing through the guiding block and the lower side of the sleeves.

For further understanding of the invention, reference is made to the following detailed description illustrating the embodiments and examples of the invention. The description is only for illustrating the invention and is not intended to be considered limiting of the scope of the claim.

### BRIEF DESCRIPTION OF THE DRAWINGS

The drawings included herein provide a further understanding of the invention. A brief introduction of the drawings is as follows:

FIG. 1 is an exploded perspective view of the present invention;

FIG. 2 is an assembly perspective view of the present invention;

FIG. 3 is an assembly perspective view of the present invention fitted with bone screws;

FIG. 4 is a cross-sectional view of the first using method of the present invention; and

FIG. 5 is a cross-sectional view of the second using method of the present invention.

### DETAILED DESCRIPTION OF THE PREFERRED EMBODIMENTS

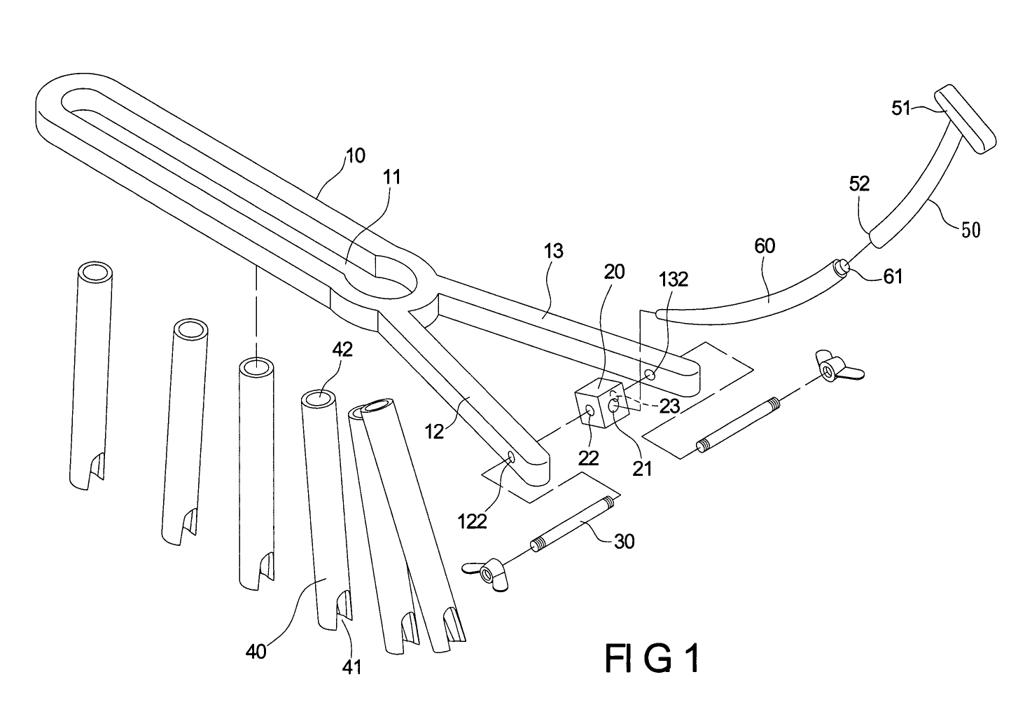

Reference is made to FIGS. 1, 2, 3, 4 and 5. The auxiliary apparatus for a vertebra surgical operation includes an auxiliary part 10, a guiding block 20, a fixing rod 30, a plurality of sleeves 40, a puncturing part 50, and a linking rod 60.

The auxiliary part 10 has an elliptic shape and includes a regulating opening 11. A first rod 12 and a second rod 13 extend backward from the edge of the two sides of the auxiliary part 10. The first rod 12 and the second rod 13 both have a first fixing hole 122 and a second fixing hole 132. The first fixing hole 122 and the second fixing hole 132 are respectively located at one end of the first rod 12 and one end of the second rod 13.

The guiding block 20 is a cube. An arc-shaped guiding groove 21 passes through the interior of the guiding block 20. A third fixing hole 22 and a fourth fixing hole 23 are respectively located at the two side surfaces of the guiding block 20. The third fixing hole 22 and a fourth fixing hole 23 are indented to the two side surfaces of the guiding block 20. The first fixing hole 122 corresponds to the third fixing hole 22. The second fixing hole 132 corresponds to the fourth fixing hole 23. The guiding block 20 is pivotably fixed to the rear side of the auxiliary part 10 via a fixing rod 30. Therefore, the guiding block 20 can be rotated to any desired angle.

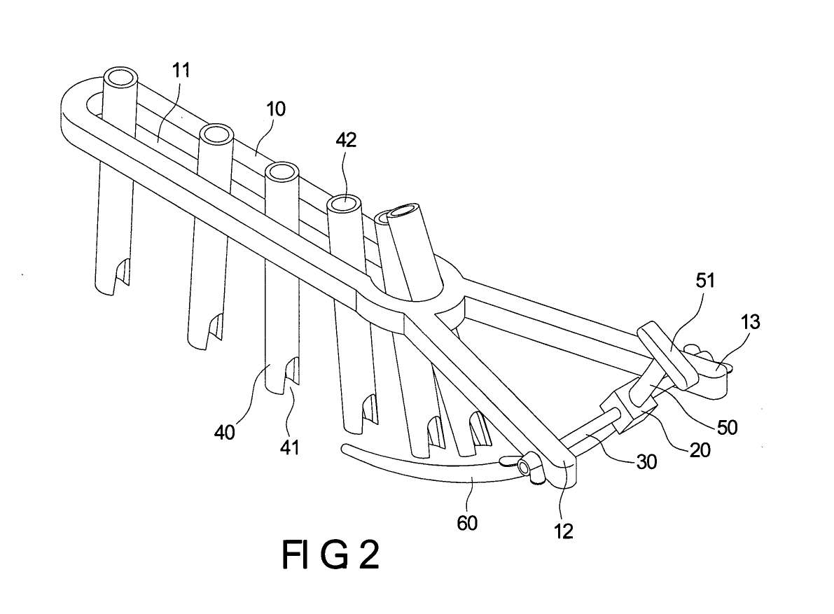

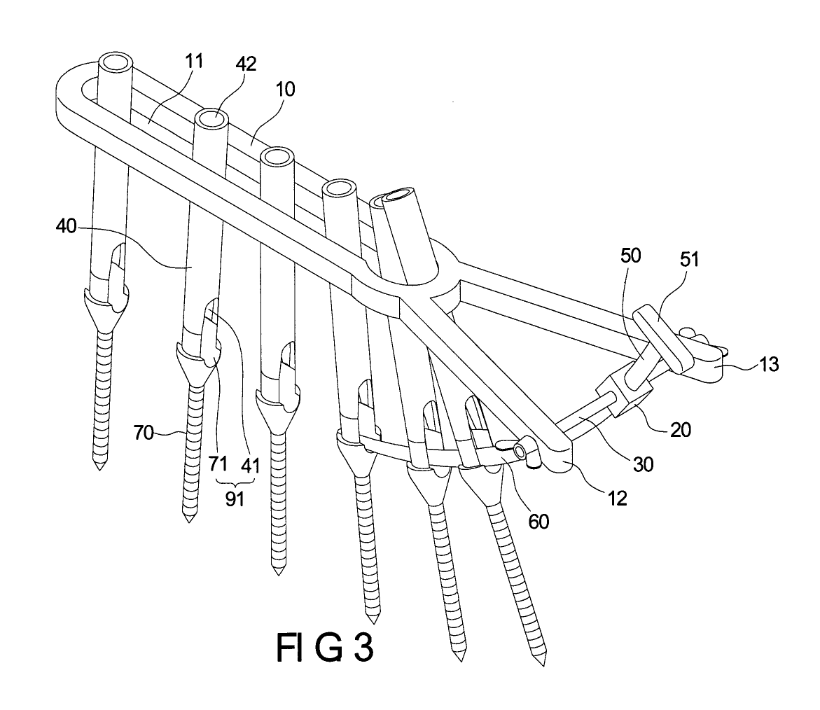

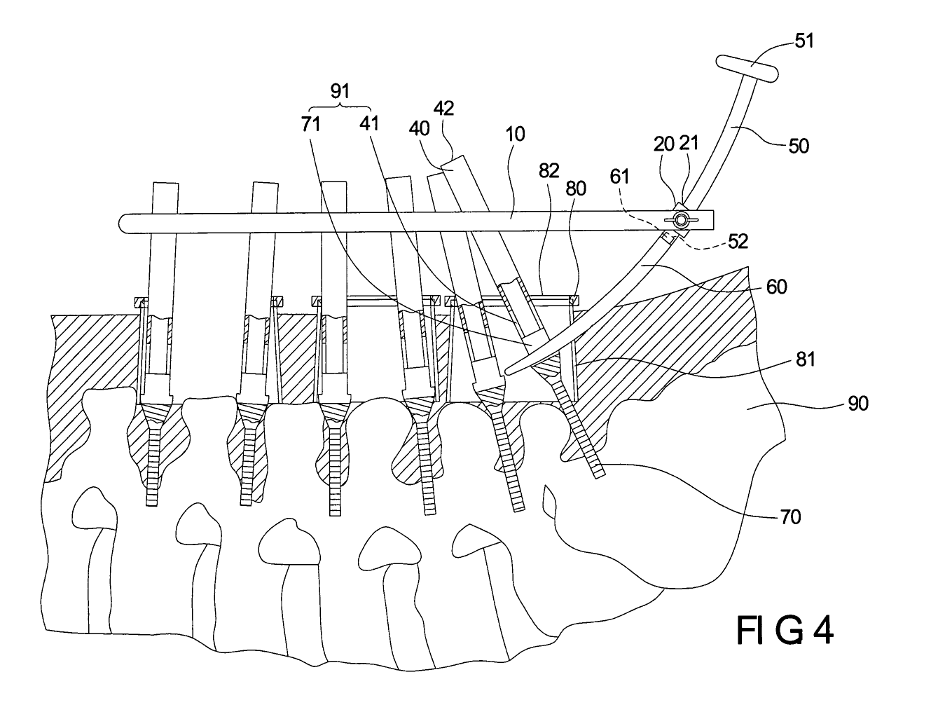

The sleeves 40 are circular sleeves. The sleeves 40 are regulated by the regulating opening 11 (as shown in FIG. 2). One end of each of the sleeves 40 has a concave groove 41. One end of each sleeve 40 is individually sleeved to an end of a bone screw 70 (as shown in FIG. 3). The bone screws are screwed to the vertebra 90 (as shown in FIG. 4). Each end of the bone screw 70 has a plow groove 71. The plow groove 71 corresponds to the concave groove 41 to form a through hole 91 (as shown in FIG. 3).

The sleeves 40 are individually placed into a plurality of expanding parts 80 (as shown in FIG. 4). The expanding part 80 is a circular hollow column. An opening 81 is formed around the fringe of the expanding part 80. The expanding parts correspond to the vertebra 90 and are placed into the muscle. The expanding parts 80 form a plurality of operating troughs 82 on the human body. The surgeon executes a vertebra surgical operation via the operating troughs 82 formed by the expanding part 80.

The puncturing part 50 is an arc cylinder rod. One end of the puncturing part 50 has a handling part 51, and a second end of the puncturing part 50 has a wedging part 52. One end of the linking rod 60 has a connecting part 61. The connecting part 61 matches the wedging part 52 to connect the linking rod 60 with the puncturing part 50. The puncturing part 50 is connected with the linking rod 60 and passes through the arc-shaped guiding groove 21. When a second end of the linking rod 60 punctures into the muscle and passes through the opening 81 of the first expanding part 80 and the first through hole 91 (as shown in FIG. 4), the first sleeve 40 is adjusted to match the arc-shaped guiding groove 21. After the sleeve 40 is adjusted, the position of the first sleeve 40 and guiding block 20 are fixed so as to guide the puncturing part 50 with the linking rod 60 to continually pass through the openings 81 of the expanding parts 80 and the through holes 91. Thereby, the linking rod 60 is received in the plow grooves 71 of the bone screws 70.

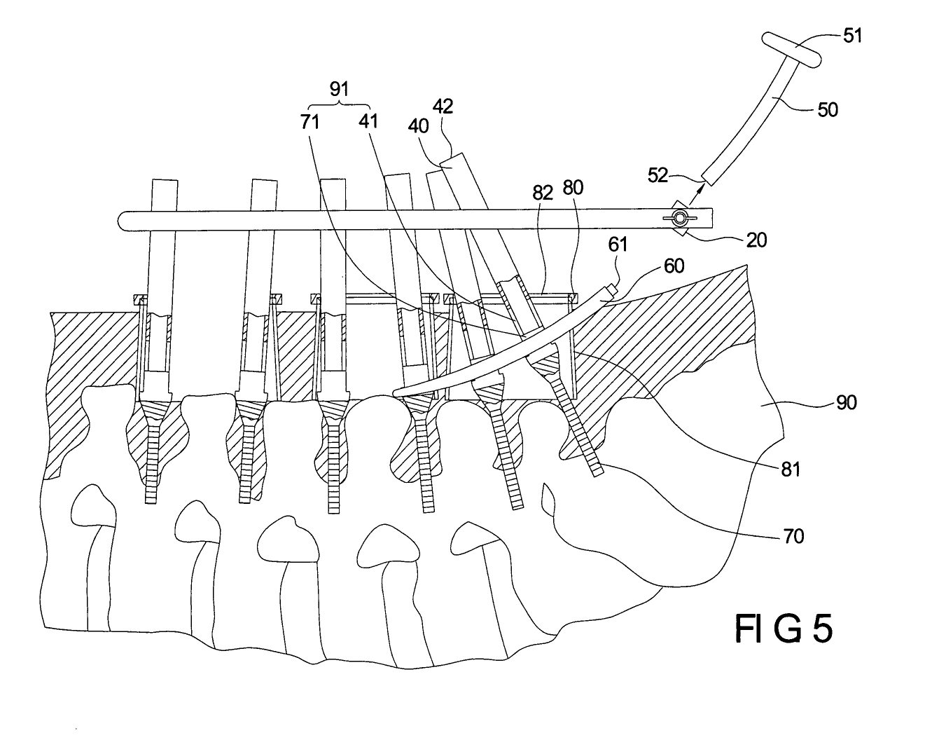

Reference is made to FIG. 5, which shows a cross-sectional view of the second using status of the present invention. When the puncturing part 50 passes through the arc-shaped guiding groove 21, and a second end of the linking rod 60 punctures into the muscle and passes through the openings 81 of the expanding parts 80 and the through holes 91, the linking rod 60 is received in the plow grooves 71 of the bone screws 70. At this time, the handling part 51 of the puncturing part 50 is rotated to make the linking rod 60 escape from the puncturing part 50. The surgeon can then check the status of the linking rod 60 received in the plow grooves 71 via the operating well 42 formed by the sleeves 40. If the linking rod 60 is not received in the plow grooves 71 in a proper position, a tool is used to push the linking rod 60 into the plow grooves 71 via the operating well 42 formed by the sleeves 40. Therefore, the linking rod 60 is fastened onto the bone screws 70 and connected with the bone screws 70 to correct and fix an injured or bent vertebra.

The present invention assists the surgeon to execute a small incision vertebra surgical operation. The goals of the small incision surgical operation are achieved, specifically making a small incision that will heal quickly.

The description above only illustrates specific embodiments and examples of the invention. The invention should therefore cover various modifications and variations made to the herein-described structure and operations of the invention, provided they fall within the scope of the invention as defined in the following appended claims.

What is claimed is:

1. An auxiliary apparatus for a vertebra surgical operation, that is applied to a small incision vertebra surgical operation, comprising:
an auxiliary part having a regulating opening;
a guiding block attached on a rear side of the auxiliary part;
a plurality of sleeves regulated by the regulating opening; and
a puncturing part passing through the guiding block and the lower side of the sleeves.

2. The auxiliary apparatus for a vertebra surgical operation as claimed in claim 1, wherein two rods both extend backwards from the two side edges of the auxiliary part, and the guiding block is pivoted between the rods.

3. The auxiliary apparatus for a vertebra surgical operation as claimed in claim 1, wherein an arc-shaped guiding groove passes through the interior of the guiding block.

4. The auxiliary apparatus for a vertebra surgical operation as claimed in claim 1, wherein the sleeves are individually placed into a plurality of expanding parts, the expanding parts correspond to the vertebra 90 and are placed into the muscle, and the expanding parts form a plurality of operating troughs on the human body.

5. The auxiliary apparatus for a vertebra surgical operation as claimed in claim 1, wherein one end of the sleeves respectively has a concave groove, one end of each of the sleeves is sleeved to one end of a bone screw, the bone screws are screwed to the vertebra, one end of the bone screw has a plow groove, and the plow groove corresponds to the concave groove to form a through hole.

6. The auxiliary apparatus for a vertebra surgical operation as claimed in claim 1, wherein one end of the puncturing part has a handling part and another end of the puncturing part is connected with a linking rod, the puncturing part passes through an arc-shaped guiding groove, and another end of the linking rod punctures into the muscle and passes through the opening of the expanding parts and the through holes to receive the linking rod in the plow grooves of the bone screws.
